(19) United States
(12) Patent Application Publication
(10) Pub. No.: US 2026/0199712 A1
(43) Pub. Date: Jul. 16, 2026

(54) AXIALLY STEERED BOILING HISTOTRIPSY

(71) Applicant: UNIVERSITY OF WASHINGTON, Seattle, WA (US)

(72) Inventors: Gilles Pierre Loic THOMAS, Seattle, WA (US); Vera A. KHOKHLOVA, Seattle, WA (US); Tatiana D. KHOKHLOVA, Seattle, WA (US); Oleg A. SAPOZHNIKOV, Seattle, WA (US)

(73) Assignee: University of Washington, Seattle, WA (US)

(21) Appl. No.: 19/136,218

(22) PCT Filed: Dec. 5, 2023
(86) PCT No.: PCT/US2023/082471

§ 371 Date: Jun. 5, 2025

Related U.S. Application Data
(60) Provisional application No. 63/430,792, filed on Dec. 7, 2022.

(51) Int. Cl. A61N 7/02; A61N 7/00

(52) U.S. Cl. CPC A61N 7/02; A61N 2007/0039

(57) ABSTRACT

A method for an ultrasound treatment using high intensity focused ultrasound (HIFU), the method including generating boiling histotripsy (BH) therapy ultrasound with a therapy transducer, applying the therapy ultrasound by directing a plurality of ultrasound pulses having ultrasound shock waves to a target tissue, generating at least one μm-scale vapor bubble at the target tissue within each pulse, growing the at least one vapor bubble to at least one mm-scale bubble, moving a focal point during each pulse of the plurality of pulses from a first region of the target tissue to a second region of the target tissue, and mechanically disintegrating the target tissue by interactions between the ultrasound shock waves with the at least one mm-scale bubble and bubble clouds, wherein the interactions take place within a duration of individual ultrasound pulses of the plurality of ultrasound pulses.

### CROSS-REFERENCES TO RELATED APPLICATIONS

This application claims the benefit of U.S. Provisional Patent Application No. 63/430,792, filed Dec. 7, 2022, the disclosures of which is hereby incorporated by reference in its entirety.

### STATEMENT OF GOVERNMENT LICENSE RIGHTS

This invention was made with government support under Grant Nos. R01EB007643 and R01EB025187 and R01GM122859, awarded by the National Institutes of Health. The government has certain rights in the invention.

### BACKGROUND

Boiling Histotripsy (BH) is a high intensity focused ultrasound (HIFU) method to generate mechanical fractionation of tissue via a combination of complex physical processes that include the localized heating of tissue by shock waves to boiling temperature at the focus, formation of a vapor cavity, interaction of incoming shock waves with a vapor cavity, scattering of shocks from the cavity, generation of bubble clouds in front of the cavity, ejection and atomization of tissue debris into the cavity, and destruction of tissue by this internally cavitating bubble cloud, microjetting, and atomization.

Because in BH large vapor bubbles are created at the focus, shock scattering contributes to the creation of the lesion. The bubble cloud generated by the shock scattering starts at the first vapor bubble formed in the focal region of the BH beam and extends towards the transducer.

However, the reported volumetric ablation speed of BH with a standard 10 ms pulse is up to 10 cc/h for the in vivo treatment of liver tissue, which is relatively low in comparison with thermal HIFU ablation that has a wider range of ablation speed up to 150 cc/h for in vivo liver treatment.

As such, BH treatment volumetric ablation rates need to be improved in order to be a viable clinical alternative to other noninvasive methods. Enhancement of a shock-scattering mechanism can contribute to accelerating of the BH ablation rate.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, disclosed herein is a method for an ultrasound treatment using high intensity focused ultrasound (HIFU), the method including generating boiling histotripsy (BH) therapy ultrasound with a therapy transducer, applying the therapy ultrasound by directing a plurality of ultrasound pulses having ultrasound shock waves to a target tissue, generating at least one μm-scale vapor bubble at the target tissue within each pulse, growing the at least one vapor bubble to at least one mm-scale bubble, moving a focal point during each pulse of the plurality of pulses from a first region of the target tissue to a second region of the target tissue, and mechanically disintegrating the target tissue by interactions between the ultrasound shock waves with the at least one mm-scale bubble and bubble clouds, wherein the interactions take place within a duration of individual ultrasound pulses of the plurality of ultrasound pulses.

In some embodiments, the plurality of pulses has a duration of 1-20 ms. In some embodiments, the therapy transducer operates in a frequency range of 1 MHz to 20 MHz. In some embodiments, the therapy transducer is moved between a plurality of regions, including the first region and the second region.

In some embodiments, the first region is closer to the therapy transducer than the second region. In some embodiments, the second region is closer to the therapy transducer than the first region.

In some embodiments, a shock amplitude at the focal point of the therapy transducer is in a range of 40 MPa to 250 MPa. In some embodiments, the plurality of ultrasound pulses is applied at a focal depth of 0.25 cm to 15 cm.

In some embodiments, the focal point is moved axially from the first region to the second region. In some embodiments, the focal point is moved at an angle. In some embodiments, the angle is about 0 to 35 degrees.

In some embodiments, a Δz of the movement of the focal point ranges from 100 to 800 μm.

In another aspect, disclosed herein is a method for an ultrasound treatment using high intensity focused ultrasound (HIFU), the method including generating boiling histotripsy (BH) therapy ultrasound with a therapy transducer, applying the therapy ultrasound by directing a plurality of ultrasound pulses having ultrasound shock fronts to a target tissue, generating at least one μm-scale vapor bubble at the target tissue during each pulse, growing the at least one vapor bubble to at least one mm-scale bubble, moving a focal point of each pulse of the plurality of pulses between a plurality of regions of the target tissue, and mechanically disintegrating the target tissue by interactions between the ultrasound shock waves with the at least one mm-scale bubble and bubble clouds, wherein the interactions take place within a duration of individual ultrasound pulses of the plurality of ultrasound pulses.

In some embodiments, the plurality of regions is an array of regions corresponding to the target tissue. In some embodiments, the method further includes sequencing each region in the array of regions, and moving the focal point between each region of the array based on the sequence. In some embodiments, the method further includes applying a number of pulses at each region of the array of regions, wherein the number of pulses differs between each region of the array of regions.

In some embodiments, the focal point is moved axially. In some embodiments, a shock amplitude at the focal point of the therapy transducer is in a range of 40 MPa to 250 MPa. In some embodiments, the plurality of ultrasound pulses is applied at a focal depth of 0.25 cm to 15 cm. In some embodiments, the focal point is moved at an angle.

### DESCRIPTION OF THE DRAWINGS

The foregoing aspects and many of the attendant advantages of this technology will become more readily appreciated as the same become better understood by reference to the following detailed description, when taken in conjunction with the accompanying drawings, wherein:

FIGS. 1A-1B are process steps of conventional boiling histotripsy;

FIGS. 2A-2H are images of conventional boiling histotripsy in transparent tissue-mimicking gel (HIFU is from the left);

FIGS. 3A-3E are process steps of steered boiling histotripsy (BH), in accordance with the present technology;

FIG. 4A is a graph of high intensity focused ultrasound (HIFU) sequence of pulses in accordance with the present technology;

FIG. 4B is an example set up for capturing images of steered boiling histotripsy (BH), in accordance with the present technology;

FIGS. 5A-5H show images of steered BH (HIFU is from the top), in accordance with the present technology;

FIGS. 6A-6B are graphs of normalized passive cavitation detection (PCD) signals recorded by a central element on the imaging probe during steered BH, in accordance with the present technology;

FIG. 7A-7B are example treatment planes, in accordance with the present technology;

FIGS. 8A-8D are photographs of lesions in ex vivo bovine heart tissue using different values of Δz (HIFU is from the right), in accordance with the present technology;

FIGS. 9A-9C are graphs acoustic pressure characteristics of the BH pulse in the focal region, in accordance with the present technology;

FIG. 10 is a photograph of steered BH in polyacrylamide (PA) gel with an 800 μm step and a 15 degree angle in accordance with the present technology;

FIG. 11 is another photograph of steered BH in PA gel with an 800 μm step and a 15 degree angle in accordance with the present technology; and

FIG. 12 is an example method for an ultrasound treatment using steered BH HIFU therapy, in accordance with the present technology.

### DETAILED DESCRIPTION

Boiling histotripsy (BH) is a pulsed high intensity focused ultrasound (HIFU) method relying on the generation of high amplitude shocks and bubble activity in the focal region to induce tissue liquefaction. A sequence of pulses, 1-20 ms long, generates boiling bubbles at the focus of the HIFU transducer within each pulse, and the remainder of the pulse then interacts with those bubbles. One effect is the creation of a prefocal bubble cloud due to shock scattering from initially generated boiling bubbles: the shock is inverted when reflected from the bubble wall resulting in sufficient negative pressure to reach intrinsic cavitation threshold immediately proximally to these bubbles. Here, a methodology is disclosed to extend the length of this prefocal bubble cloud by steering the focus toward the transducer during each BH pulse and thus accelerate treatment.

In an exemplary embodiment of the method, a BH system comprising a 1.5 MHz 256-element phased array connected to a Verasonics V1 system was used. High-speed imaging in transparent gels was performed to observe the extension of the bubble cloud resulting from shock scattering. Volumetric BH lesions were generated in ex vivo tissue. Results showed a threefold increase of the volumetric ablation rate with focus steering compared to standard BH.

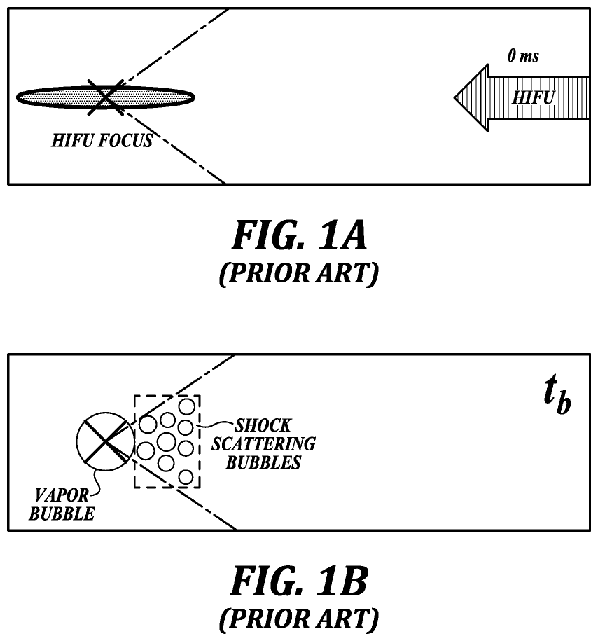

FIGS. 1A-1B are process steps of conventional boiling histotripsy. Boiling histotripsy (BH) is a high intensity focused ultrasound (HIFU) method to generate mechanical fractionation of tissue via a combination of complex physical processes that include the localized heating of tissue by shock waves to boiling temperature at the focus, formation of a vapor cavity, interaction of incoming shock waves with a vapor cavity, scattering of shocks from the cavity, generation of bubble clouds in front of the cavity (as shown in FIG. 1B), ejection and atomization of tissue debris into the cavity, and destruction of tissue by this internally cavitating bubble cloud, microjetting, and atomization. Lesions created by this method have sharp margins between the treated and untreated tissue, while causing negligible thermal injury to the surrounding tissue. BH treatments can be safely delivered transcutaneously and partially transcostally, allowing noninvasive ablation of targets in the liver and kidneys, the treatments of abscesses, the liquefaction of hematomas, and the ablation of prostate tissue via transrectal approach.

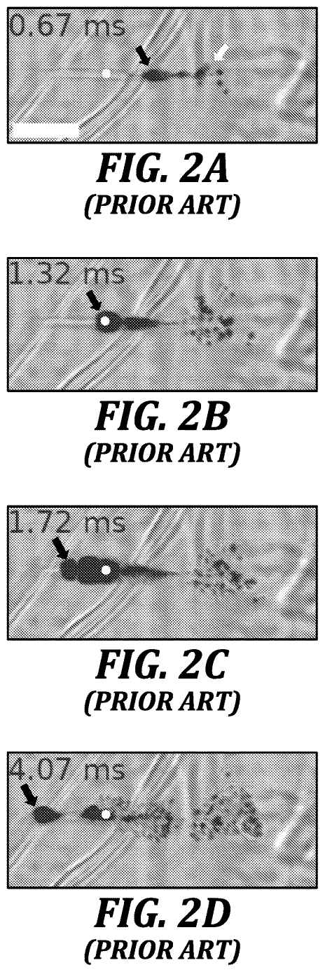

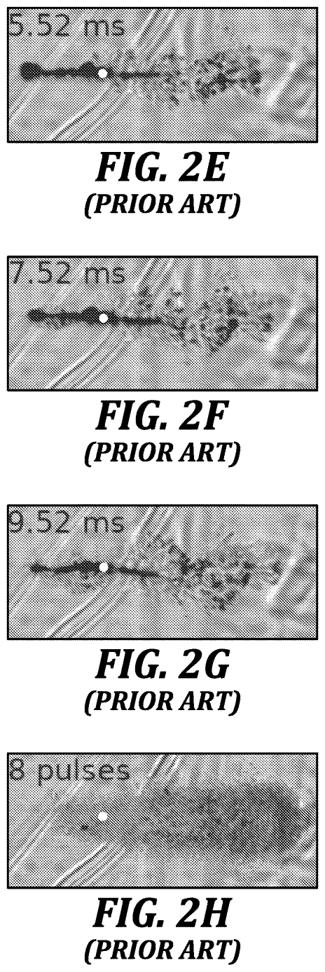

FIGS. 2A-2H are images of conventional boiling histotripsy. FIGS. 2A-2G show a treatment with a standard, i.e., fixed focus position, represented by a white dot, one 10 ms BH pulse with imaging at 20,000 frames per second (fps). The scale bar represents 2 mm, and the BH pulse was incident from the right side of the images. At 0.67 ms post BH exposure, a vapor bubble (black arrow) with prefocal cavitation (white arrow) due to shock scattering is visible. Secondary vapor bubbles (black arrows) appear behind the first one at 1.32, 1.72, and 4.07 ms, respectively. FIG. 2H is a photograph of a lesion as seen in PA with HIFU off after eight pulses with a 0.0167 Hz PRF (1 pulse/minute).

An example of high-speed imaging at 20 kfps of a standard 10 ms BH treatment with a 0.0167 Hz PRF (1 pulse/min) is shown in FIGS. 2A-2H. The first pulse of the treatment generated a boiling bubble at 0.67 ms, with a visible shock scattering bubble cloud in front of it, i.e., toward the transducer. Diffraction at the vapor bubble and the prefocal bubble cloud allowed for more boiling bubbles to appear behind previous vapor bubbles after 1.32, 1.72, and 4.07 ms of the BH exposure. During the 10 ms of each BH pulse, a prefocal bubble cloud generated by the shock scattering effect, as well as boiling bubbles themselves, were in constant movement-pushed along the BH transducer axis while also moving randomly in the transverse direction due to the acoustic radiation force exerted on them. This resulted in a slow growth of the cavitation bubble cloud in front of the boiling cavity toward the BH transducer, as well as perpendicularly to the transducer axis.

Bubbles appearing from the side of the vapor cavity could also be seen starting from 1.72 ms. After 5.52 ms, the radius of the vapor bubbles was slowly decreasing as a result of the shielding of the now large prefocal bubble cloud, with the largest vapor bubble losing around 66% of its radius, but not to the point of not being visible by the end of the 10 ms pulse.

After delivering of a total number of eight BH pulses, the area in the PA gel damaged by cavitation had a comet shape-a dark, almost circular head with a gradually lighter shade trail behind it, with a total length of 6 mm and maximum width of 1.9 mm. A notable particularity during this treatment and visible in the supplemental video was that at the third and fifth pulse no boiling happened, but instead shock scattering cavitation started immediately when the BH pulse reached the focus due to bubble nuclei still present there. In the sixth pulse, the same shock scattering cavitation also started immediately; however, boiling can be seen happening moments later behind the prefocal cavitation. While boiling was visible at 0.67 ms in the first BH pulse, it would happen later in the following pulses: 0.77 ms for the second pulse, 0.92 ms for the fourth pulse, 1.32 ms for the seventh pulse, and 1.52 ms for the eighth and last pulse. This treatment was representative of others' treatments in PA, where all those phenomena would happen as well. As mentioned above, conventional BH can take extended amounts of time. Accordingly, improvements of BH are needed.

In one aspect, described herein is a method that uses millisecond-long—from 1 to 20 ms—pulses with high-amplitude shock fronts, delivered at a low duty cycle of 1-2%. In each pulse, the highly nonlinear acoustic wave at the focus heats the tissue up to the boiling point, resulting in the creation of mm-sized vapor bubbles. The remainder of the pulse then interacts with these bubbles, resulting in a tadpole-shaped lesion. BH differs from shock-scattering cavitation histotripsy, in which the pulse is usually only a few-around 5 to 20-cycles long, also delivered at similar duty cycle. Generation of large bubble clouds in this case relies the initial presence of cavitation nuclei, on scattering and inversion of the shock fronts of each wave cycle off the surface of the generated cavitation bubbles from the previous wave cycle. Bubble clouds can be also generated without the requirement of the presence of shock fronts and cavitation nuclei if the peak negative pressure at the focus is very high, exceeding the intrinsic cavitation threshold of about −28 MPa (microtripsy).

Because in BH large vapor bubbles are created at the focus, shock scattering contributes to the creation of the lesion. The bubble cloud generated by shock scattering starts at the first vapor bubble formed in the focal region of the BH beam and extends towards the transducer. The incoming BH waves diffract on the existing vapor bubble combined with the prefocal bubble cloud formed by shock scattering and may generate additional vapor bubbles behind the initial one, i.e., distal to the focus.

The axial size of the bubble cloud resulting from shock scattering generated in a standard BH treatment is limited by the prefocal point where the sum of the incident and reflected pressures is not sufficient to exceed the intrinsic cavitation threshold. This bubble cloud is thought to be responsible for the proximal part of the BH lesions, i.e., the head of their tadpole shape. The size of the BH lesion is thus limited in a similar manner.

Disclosed herein is a method to extend the axial dimension of this bubble cloud by moving the focus axially towards the BH transducer within the duration of each BH pulse after vapor bubbles were created, thus generating an elongated bubble cloud through shock scattering.

The feasibility of this approach was demonstrated in optically transparent tissue-mimicking gels using high-speed photography to observe the evolution of the bubble cloud during the focus steering, and to perform a limited parameter optimization. Using parameters of BH pulses that ensured the desired formation of the cavitation clouds in gel phantoms, the method was then applied to ex vivo bovine cardiac tissues to generate volumetric lesions of different sizes. The ablation rates achieved using this new approach were compared to those of a standard BH protocol without steering the focus axially during BH pulses.

In one example, the BH transducer used is a 1.5 MHz, 256-element spiral array made from composite piezoelectric material (Imasonic, Voray sur l'Ognon, France), and described in detail. Briefly, the array was spherically focused with a radius of 120 mm, had an outer diameter of 144 mm, and a central opening of 40 mm diameter for mounting an inline ultrasound imaging probe with a −6 dB bandwidth comprised between 2.2 and 5.2 MHz (3PE, Humanscan, Gyeonggido, South Korea). The elements of the array were circular with a diameter of 7 mm and arranged in 16 spiral branches with a minimum of 0.5 mm spacing between the elements. In some embodiments, the method may be carried out with any transducer with the ability to axially move the focus. In some embodiments, the transducer may be manually actuated to move or adjust the focus, but in other embodiments, the transducer may move the focus automatically.

The array was electrically matched and connected to a 4-board Verasonics V1 system (V-1 Ultrasound Acquisition platform, Verasonics Inc, Kirkland, WA, USA) with HIFU option. The Verasonics system was modified by adding seven electrolytic capacitors (B41560A9159M000, EPCOS, Munich, Germany) identical to the internal DC power supply in parallel with the external 1200 W DC power supply from the HIFU option (QPX600DP, Aim-TTI, Huntingdon, U.K.).

This BH system has been fully characterized in nonlinear shock-forming regimes and at acoustic power outputs relevant to BH in a previous publication. Specifically, boiling could be reached in polyacrylamide gels and ex vivo tissues with a 10 ms BH pulse at the acoustic power of 251 W. At this power, the peak positive pressure was 96 MPa and the peak negative pressure was-17 MPa, and the −6 dB focal length was 3.7 mm and its width was 0.32 mm. When used for volumetric BH lesions, the electronic steering of the system was limited to a maximum of +6 mm transversely and +6 mm axially around the geometric focus.

The inline ultrasound imaging probe was connected to a separate 2-board Verasonics V1 system. The B-mode imaging rate was 27 Hz, and during each BH pulse the probe was passively recording at a sampling rate of 18 MHz the backscattered BH waves and broadband noise emissions from cavitation bubbles, thus serving for passive cavitation detection (PCD).

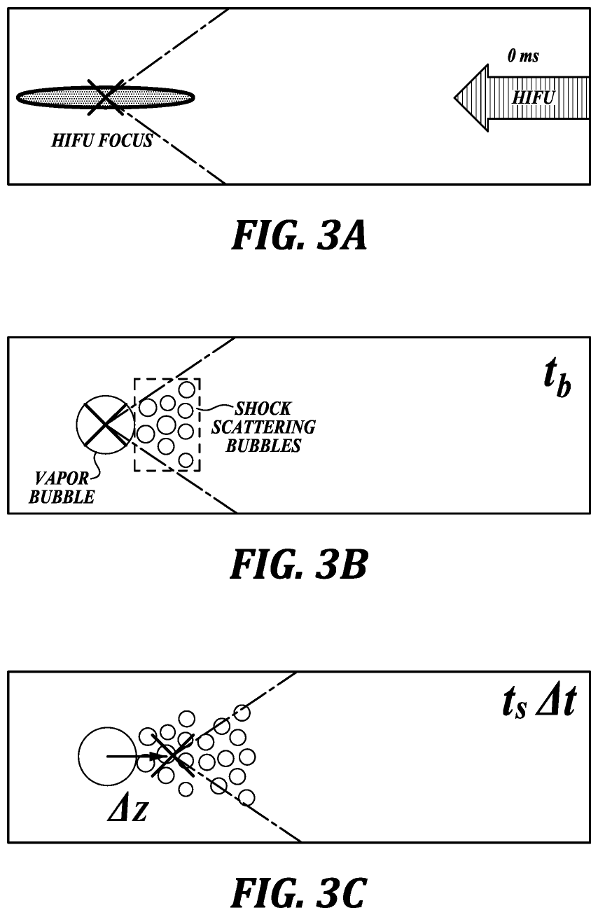

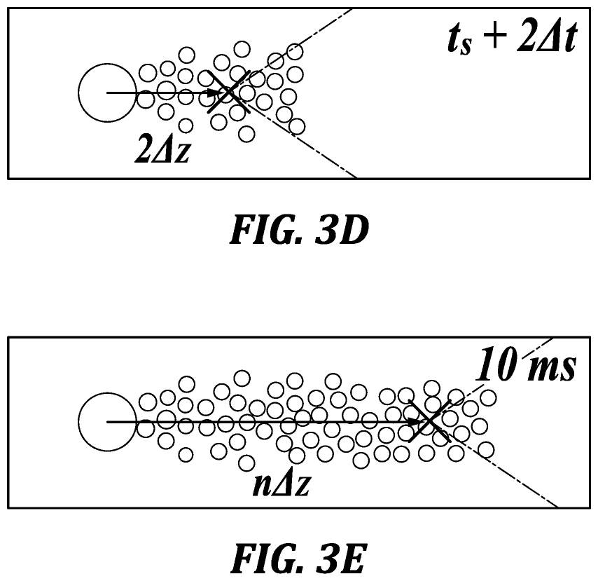

FIGS. 3A-3E show process steps of steered boiling histotripsy (BH), in accordance with the present technology. The concept of a modified BH method described herein work is illustrated in FIGS. 3A-3E for a 10 ms BH pulse. The black cross corresponds to the transducer array focus position.

In one aspect, disclosed herein is a method for an ultrasound treatment using high intensity focused ultrasound (HIFU), the method including generating boiling histotripsy (BH) therapy ultrasound with a therapy transducer, applying the therapy ultrasound by directing a plurality of ultrasound pulses having shock fronts to a target tissue, generating at least one μm-scale vapor bubble at the target tissue within each pulse, growing the at least one vapor bubble to at least one mm-scale bubble, moving a focal point of each pulse from a first region of the target tissue to a second region of the target tissue during the pulse, and mechanically disintegrating the target tissue by interactions between the at least one mm-scale bubble and the ultrasound shock waves, wherein the interactions take place within a duration of each individual ultrasound pulse of the plurality of ultrasound pulses.

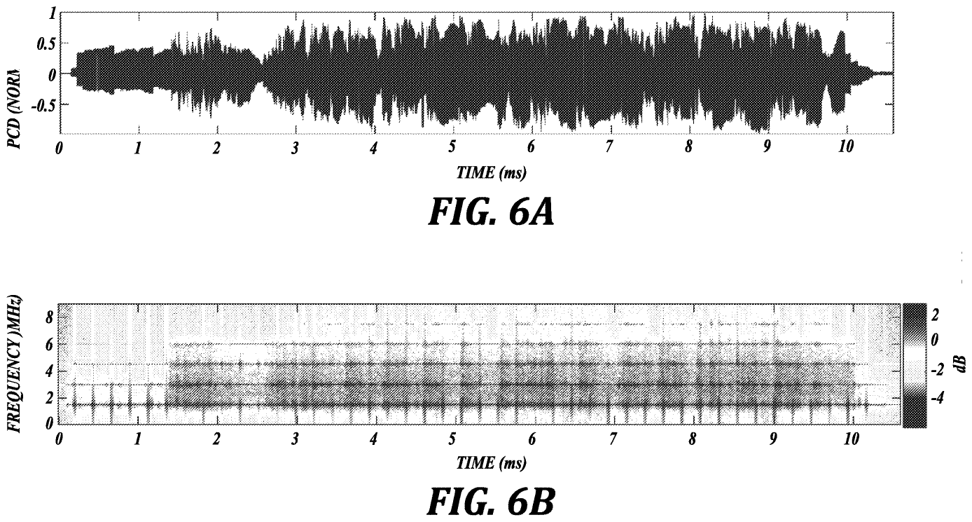

First, in FIG. 3A, a strongly shocked HIFU at the focus heats a small region up to boiling temperature. In FIG. 3B, a vapor bubble is generated at the focus within the length of the BH pulse. At time-to-boil (tb), a vapor bubble is created and subsequent interactions from the BH pulse generates an initial layer of bubble cloud prefocally as a result of shock scattering. Experimentally, the tb was determined by looking at the transducer array backscattered signal—here recorded by the ultrasound imaging probe and referenced as PCD signal—as when boiling bubbles were created the PCD signal becomes noticeably noisier, with a substantial increase of broadband noise and harmonics level as a result of the cavitation at the focus, as shown in FIGS. 6A-6B. As there was a certain variability in the tb, it was verified to be less than 80% of the steering starting time (ts) during a treatment at 5 different positions within each pulse.

As such, in FIG. 3B, the acoustic power required to reach boiling in the gel or tissue before the steering starting time ts is found. The pressure level at the focus for that acoustic power was then obtained from previous characterization of that BH transducer array. In FIG. 3D, at ts, the focus is electronically steered toward the transducer array with a step Δz. Once the vapor cavity is created, reflection of the shocks is initiated generating an initial layer of a bubble cloud prefocally. In FIG. 3C, at ts, when this first layer of bubbles had already been formed, the focus was electronically steered prefocally—i.e., toward the transducer array—with a step Δz. This results in the creation of a new layer of bubbles by shock scattering of the shocks in the BH pulse off of the previously created bubble cloud. This cloud originated at the proximal side of the previous bubble cloud and extended it further toward the transducer. Shock scattering interactions with the previously created bubble cloud generates a new layer of bubble cloud, as shown in FIGS. 3D and 3E.

The previous steps (illustrated in FIGS. 3D and 3E) are repeated n times for every time step Δt until the end of the BH pulse, here 10 ms. In some embodiments, the method may take 10 ms, but in other embodiments, the method may be longer or shorter. This process of steering the BH focus towards the transducer and thus extending the bubble cloud length through shock scattering, was repeated n times with the same Δz step for every time step Δt until the end of the 10 ms BH pulse. Because the axial steering of the BH focus played a major role in the resulting cavitation cloud, this method will be referred to as ‘steered boiling histotripsy’, or steered BH, herein.

In this method, four parameters may impact the creation of the bubble cloud and the lesion generated, namely the total length of the BH pulse, the time ts when the steering start, the spatial step Δz, and the temporal step Δt. As this example focused on the feasibility of the proposed method as an alternative for conventional BH, most of these parameters were constant. As such, the length of the BH pulse was set constant and equal to 10 ms, as it is the most commonly used value in the BH literature. The time ts depends on the value of the time-to-boil tb. These times are determined experimentally, but it was shown in previous publications that boiling could be reached in less than 1 ms. The value of the spatial step Δz was variable and its range would be verified experimentally, as if chosen too large the shock scattering effect might not happen and thus the bubble cloud will not be extended. Its maximum value was also limited by the electronic steering capabilities of the array, where here the maximum axial steering was chosen as +6 mm around the geometric focus as in previous work done with this array on BH volumetric lesions. Finally, the temporal step Δt was set as constant and equal to 0.5 ms.

In some embodiments, the plurality of pulses has a duration of 1-20 ms. In some embodiments, the therapy transducer operates in a frequency range of 1 MHz to 20 MHz. In some embodiments, the first region is a first end of the target tissue. In some embodiments, the second region is a second end of the target tissue. In some embodiments, the second region is closer to the therapy transducer than the first region. In some embodiments, a shock amplitude generated by the therapy transducer at the focus is in a range of 40 MPa to 200 MPa. Preferably, in some embodiments, the shock amplitude is between 60 MPa to 150 MPa. More preferably, in some embodiments, the shock amplitude is between 120 and 140 MPa. One skilled in the art should understand that the shock amplitude depends on the frequency and pulse duration. For higher frequencies, less shock amplitude may be applied for the same pulse duration. For a shorter pulse duration, higher shock amplitudes may be used. In some embodiments, the plurality of ultrasound pulses is applied at a focal depth of 0.25 cm to 15 cm. In some embodiments, a spatial step (Δz) of the movement of the focal point ranges from 100 to 800 μm.

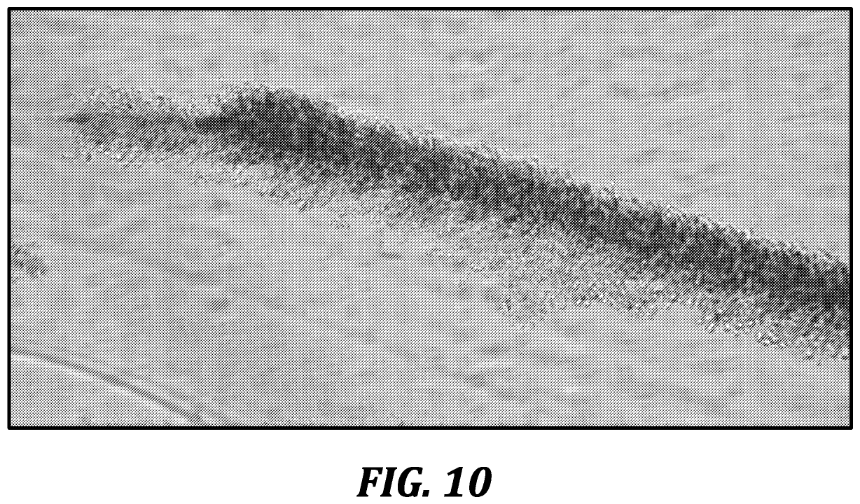

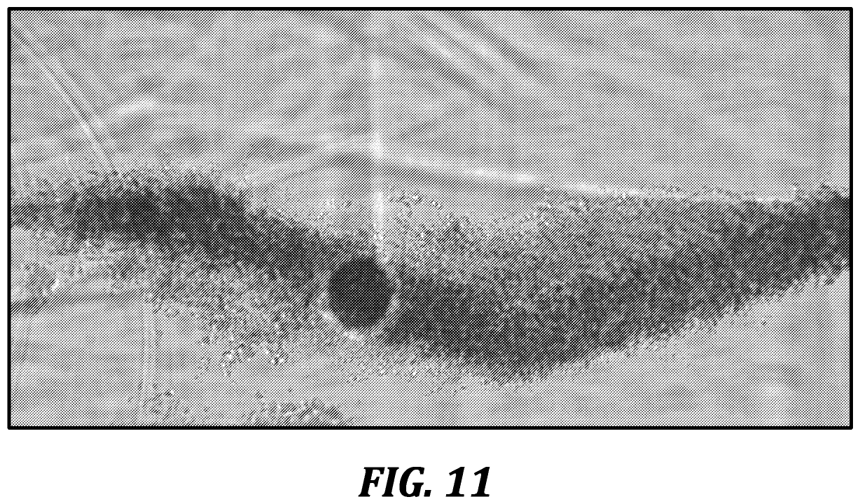

In some embodiments, the focal point is moved axially from the first region to the second region. In some embodiments, the focal point is moved at an angle. In some embodiments, the angle is between about 0 to 35 degrees, as shown in FIGS. 10 and 11. In some embodiments, the angle is measured from 0 degrees (a direct line from the focal point at the first region to the therapy transducer). In some embodiments, the angle is 15 degrees. In some embodiments, the focal point is moved from the first region to the second region a plurality of times.

Previous work on shock scattering demonstrated that only a small number of acoustic cycles—between 5 to 20—could extend the bubble cloud to its maximum size, reaching the point where the peak negative pressure in the scattered wave does not exceed the intrinsic threshold, while BH lesions could be made with 1 ms pulse, including time-to-boil. This suggest that 0.5 ms is enough for both the extension of the bubble cloud and the generation of lesion typical to BH.

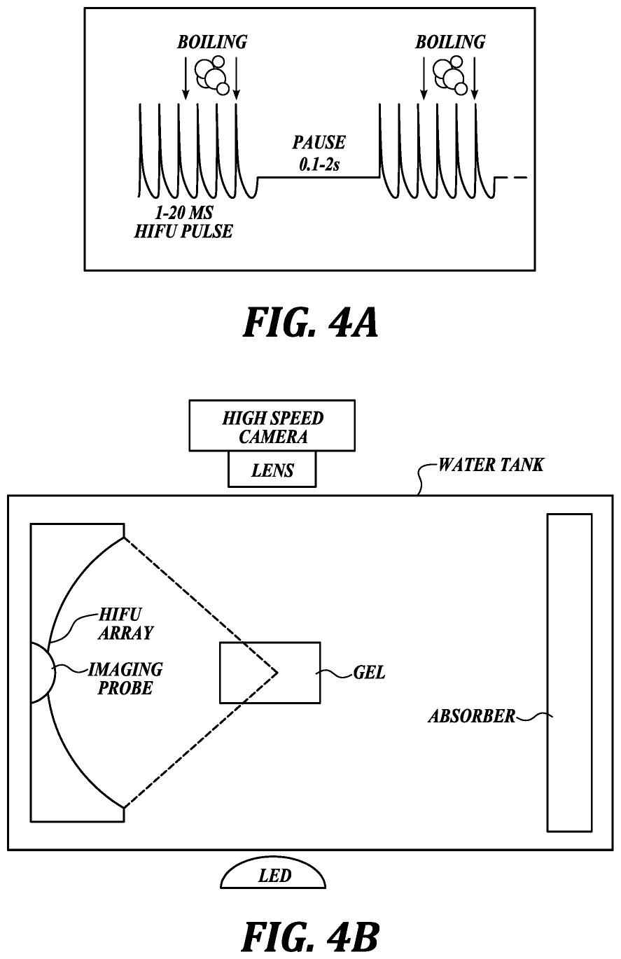

FIG. 4A is a graph of two HIFU pulses from a sequence of pulses in accordance with the present technology. In some embodiments, the pulses of HIFU may extend over 1-20 ms, followed by a pause of 0.1-2 seconds. As explained above, the ultrasound shock waves within a given pulse may cause localized boiling in the target tissue, therefore generating vapor bubbles at the focal area. Next, these vapor bubbles may rapidly grow into significantly larger vapor bubble during the same pulse in the HIFU operation. These bubbles interact with remaining shock wavefronts within the same pulse. An example of such rapid growth in the bubble size is illustrated in FIG. 4A, which shows an initially generated vapor bubble growing to a larger size.

FIG. 4B is an example set up for capturing images of steered boiling histotripsy (BH), in accordance with the present technology. High-speed photography of steered BH exposures in transparent gel phantoms was performed to directly observe the initiation of boiling and cavitation cloud formation. A diagram of the experimental setup is shown in FIG. 4A. The experiments were performed at room temperature of 20° C. in a glass-wall tank filled with deionized water, degassed to below 20% oxygen saturation with an in-house built degassing system.

The BH array was fixed on one side of the tank, and a polyurethane rubber acoustic absorber was placed on the opposite side to minimize reverberations. The gel samples were placed in a custom 3-D-printed holder and positioned in the tank using a three-axis positioning system (Velmex Inc., Bloomfield, NY, USA). A high-speed camera (Fastrax APX-RS, Photron, San Diego, CA, USA) in combination with a diffuse LED light on the other side of the tank were used to record the bubble activity resulting from the steered BH pulses inside the gel samples positioned in-between. The photographs were captured with a shutter speed of 4 μs in a plane collinear to the axis of the BH transducer array. A 105 mm lens (Nikon, Melville, NY, USA) was used to obtain a resolution of 27 μm per pixel. Recordings were made at 20 and 100 kfps with a field of view of 512×256 pixels (13.8×6.9 mm) and 256×32 pixels (6.9×0.9 mm), respectively.

Different steered BH exposures were tested in PA gel phantoms without bovine serum albumin. This gel has acoustic properties very close to tissues and allows for visualization of the vapor and shock scattering bubbles during BH sonications. After BH sonications, the areas damaged by cavitation were visibly darker, allowing for visualization of the region affected by cavitation during the treatment. The gel samples were prepared within 48 h of the experiments into plastic molds of 80×50×50 mm. The samples were then cut in half to a final size of 80×50×25 mm before being placed in the sample holder inside the water tank. For all the treatments, the geometric focus of the BH transducer array was 20 mm deep inside the gels. As a consequence of the gel samples being cut, and also due to small bubbles present at the gel/mold interface during polymerization, some artifacts could be seen on the high-speed photographs. These artifacts were purely visual, easily recognizable, and did not affect the experiment in any way.

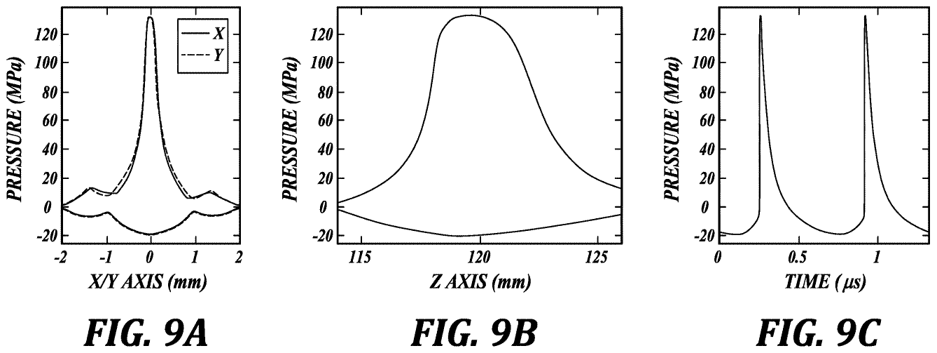

Repeatable boiling in PA gels was reached in less than 2 ms for the acoustic power of 608 W, and thus ts was set equal to 2.5 ms for all the following experiments. The focal beam shape of the peak positive and negative pressures, as well as two cycles of the focal waveform with shock fronts, which correspond to this acoustic power, were matched with a previous characterization of the transducer made using acoustic holography and nonlinear modeling and are shown in FIGS. 9A-9C.

At this power, the peak positive pressure in free field in water at the focus was 132 MPa and the peak negative pressure was-20 MPa with a shock amplitude of 136 MPa, and the dimensions of the focal lobe for the peak positive pressure at −6 dB level were 4.7 mm axially and 0.35 mm laterally (see FIGS. 9A-9C).

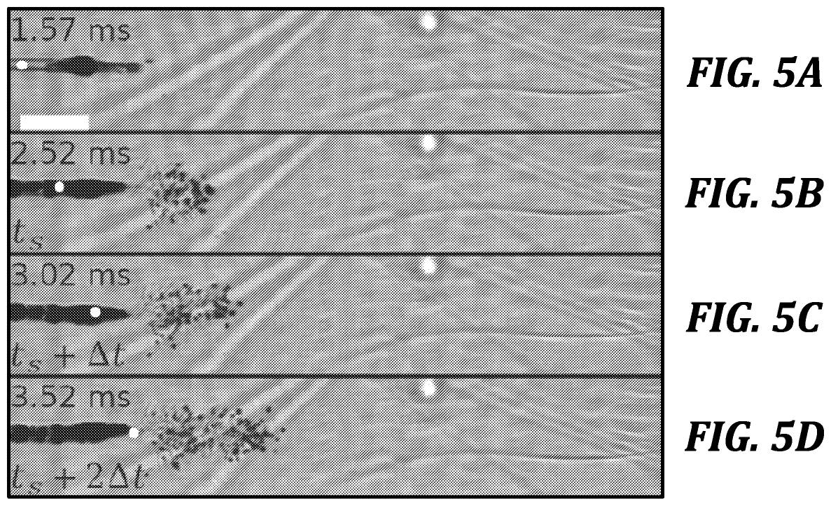

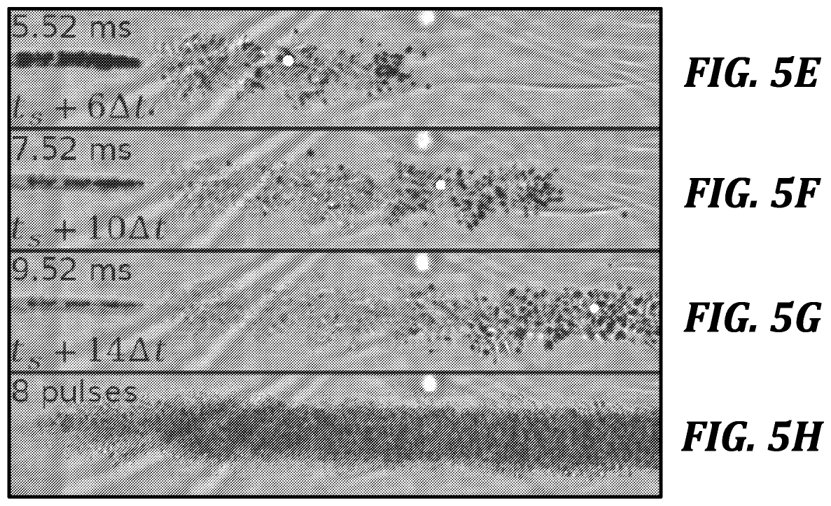

FIGS. 5A-5H are images of steered BH, in accordance with the present technology. Shown is steered BH treatment in PA gel with Δz set at 800 μm. The white bar represents 2 mm, the BH pulse was arriving from the right side, and small white dot represents the focus position. At 1.57 ms (<ts) after start of BH exposure, a vapor bubble is visible (FIG. 5A). At 2.52 (FIG. 2B), 3.02 (FIG. 2C), and 3.52 ms (FIG. 2D), respectively, the focus was steered by Δz=800 μm toward the transducer array relative to the previous figure. At 5.52 (FIG. 5E), 7.52 (FIG. 5F), and 9.52 ms (FIG. 5G), respectively, the focus had been steered four times by Δz=800 μm relative to the previous figure. FIG. 5H is the lesion as seen in PA gel with HIFU off after eight pulses.

Next, steered BH exposures with varying the parameter Δz were performed.

It was observed that a new bubble cloud resulting of shock-scattering was generated reliably with Δz value of up to 1.2 mm. However, due to steering limitations of the array and for a 10 ms pulse with a Δt value set at 0.5 ms, the maximum value of Δz was 800 μm, resulting in a total steering of 12 mm along the HIFU beam axis.

The pulse started with a focus electronically steered 6 mm behind the geometric focus, and was then moved a total of 15 times by a spatial step of 800 μm toward the HIFU transducer every temporal step of Δt=0.5 ms. During the first pulse, boiling was reached at 1.62 ms, and after each Δz spatial step, the bubble cloud was seen extending axially by values comprised between 603 and 1140 μm locally. This high variability of the growth of the bubble trail can be explained by the strong acoustic radiation force that was exerted by the BH pulse, with the region affected seen as pushed along the transducer axis, but also slightly off-axis (up and down on the photographs). The affected area would then move back progressively to its initial position as the focus was steered away from it, and later as the BH pulse was turned off. As the focus was moving further away from the initial boiling bubbles, their radius was seen progressively decreasing, some of them disappearing before the end of the 10 ms pulse. The same was observed for bubbles resulting from shock scattering.

However, bubbles that were in close proximity to the current BH focus position could be seen oscillating and collapsing, even with the prefocal shielding. This behavior could be better observed at 100 kfps. Finally, as observed earlier in standard BH, remnants of bubble nuclei from the previous BH pulse at the focus would sometimes initiate the formation of a cavitation cloud immediately at the start of the BH pulse prior to, or even on occasions without, any boiling. In those cases, with or without boiling, the shock scattering bubble trail was still created consistently. The lesion in PA had a maximum width of 1.6 mm and a total length of 15.4 mm.

Lesions smaller in the axial direction were generated by reducing the value of Δz, where its value was set to 400 μm. Similar to the previous treatments, displacement of the bubble clouds under the influence of the acoustic radiation force could be observed. However, the displacement perpendicular to the BH transducer axis was larger than for the 800 μm case, so much that in some cases it would delay the shock scattering cloud as seen at 3.02 ms, or also the next bubble cloud would start from the upper or lower part of the previous cloud, yielding this zigzag shape bubble trail seen at 9.52 ms. Also similar to the previous treatments was the progressive decrease in radius of the original boiling bubble as the focus moved away. After eight pulses, the lesion's total length was 10.6 mm. The difference of about 2 mm length between the picture at 9.52 ms for the first pulse and the picture after eight pulses showed that the acoustic radiation force pushed and compressed the gel and bubble cloud axially during the treatment. The width of the lesion was slightly larger close to the boiling area, measuring 1.8 mm there compared to 1.6 mm further prefocal, which was the same as in the case of a Δz of 800 μm. This width variability can be attributed to the accrued “wiggling” of the bubble cloud during the pulse exposure. Overall, any values of Δz that were less than 800 μm generated repeatably the bubble cloud trail.

Based on the previous results in PA gels, four different values of Δz were selected for evaluation: 0 (i.e., standardBH), 400, 600, and 800 μm. Achieving completely fractionated lesions with fixed-focus BH exposures required 8 pulses per treatment (PPP), as previously reported for bovine heart tissue; achieving complete lesions in steered BH exposures with Δz=400 μm required 10 PPP, and with Δz=600 and 800 μm required 12 PPP. Using a lower number of PPP resulted in residual connective tissue inside some of the lesion.

The bubbles resulting from each BH pulse were visible on the inline B-mode imaging right after the pulse and quickly faded away in less than a second. For a steered BH pulse with Δz >0, a line of hyperechoic bubbles was distinguishable. The length of this B-mode line would variate at each pulse. As the treatment progressed, and for all values of the spatial step Δz including fixed-focus BH with Δz=0, the likelihood of cavitation cloud forming instantly increased, which was manifested as high PCD noise level immediately at the start of BH pulse. In particular, PCD signals shown in FIGS. 6A-6B, characteristics of BH with clear backscatter before the boiling and highly noisy backscatter after the boiling were rarely observed after delivering more than 2 PPP. After about three quarters of the treatment time for steered BH, swirling movement of scatterers inside the lesions was visible on B-mode. Also, during the treatment in all cases, a hyperechoic layer was progressively seen appearing on B-mode at the distal part of the lesion. Once the HIFU was turned off, this layer slowly faded and disappeared after about 5-10 min. This behavior has been previously reported for standard BH volumetric treatments as well. The lesions appeared as hypoechoic on B-mode imaging, either with the inline imaging probe or with the high-resolution ultrasound. imaging system allowing for accurate measurements. The mean and standard deviation of the measured lesion dimensions for the different values of Δz, as well as the average ablation rate, are presented in Table I herein.

FIGS. 6A-6B are graphs of normalized PCD signals recorded by a central element on the imaging probe during steered BH, in accordance with the present technology. Shown is an example of the normalized PCD signal recorded by the central element of the inline imaging probe during a BH pulse (FIG. 6A) with its corresponding spectrogram (FIG. 6B. Boiling and subsequent reflections of nonlinear BH waves and cavitation cause a noticeable increase in broadband noise and harmonics level.

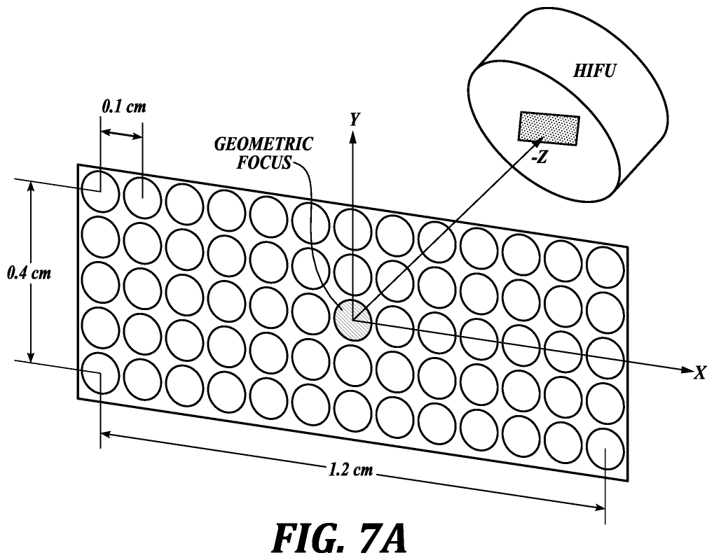

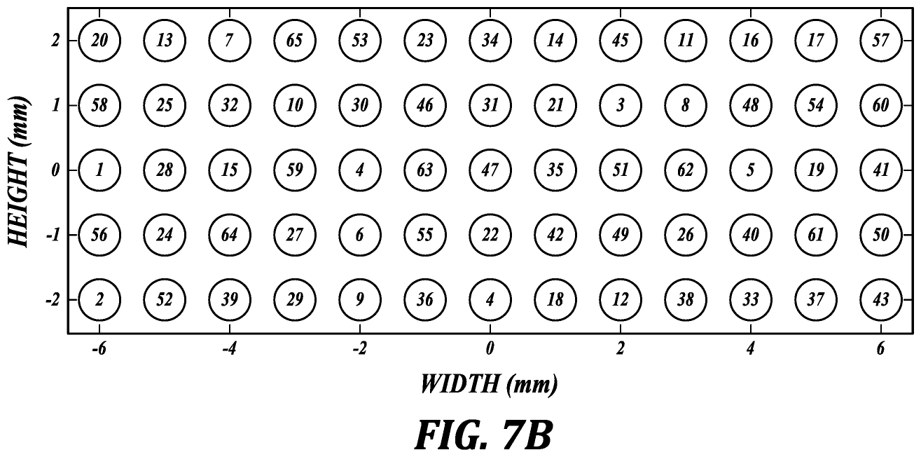

FIGS. 7A-7B are example treatment planes, in accordance with the present technology. In another aspect, disclosed herein is a method for an ultrasound treatment using HIFU, the method including generating boiling histotripsy (BH) therapy ultrasound with a therapy transducer, applying the therapy ultrasound by directing a plurality of ultrasound pulses having ultrasound shock waves to a target tissue, generating at least one μm-scale vapor bubble at the target tissue, growing the at least one vapor bubble to at least one mm-scale bubble, moving a focal point of the plurality of pulses between a plurality of regions of the target tissue during the plurality of pulses, and mechanically disintegrating the target tissue by interactions between the at least one mm-scale bubble and the ultrasound shock waves, wherein the interactions take place within a duration of individual ultrasound pulses of the plurality of ultrasound pulses.

In some embodiments, as shown in FIGS. 7A-7B, the plurality of regions is an array of regions corresponding to the target tissue. In some embodiments, the method further includes sequencing each region in the array of regions and moving the focal point between each region of the array based on the sequence. In some embodiments, the method further includes applying a number of pulses at each region of the array of regions, wherein the number of pulses differs between each region of the array of regions. In some embodiments, the focal point is moved axially.

The experimental arrangement (FIG. 7A) used for the ex vivo exposures was the same as in FIG. 4B, without the camera and light, and by replacing the gel samples with ex vivo bovine cardiac tissue samples. Bovine heart tissue was used, as it allowed to easily make gross evaluation of the BH lesions.

A fresh bovine heart was obtained from a local abattoir and kept on ice during transportation and before the experiments. The heart was cut into samples of about 70×40×40 mm size, and any pericardial fat was removed from the samples if present. The heart tissue samples were then placed into cold degassed saline solution and degassed in a desiccant chamber for over an hour. Agarose solutions were prepared in parallel by mixing agarose powder (UltraPure Agarose, Invitrogen) into deionized water (1.5% wt./vol agarose/water) and was then boiled in a microwave oven to displace any dissolved gases present. The agarose solution was then poured into plastic molds of 80×50×50 mm and left to cool. Once the temperature of the agarose solution reached 40° C., the tissue samples were placed into the molds and left to polymerize in a cold-water bath for about 30 min.

The acoustic power required to reliably reach boiling in the bovine heart tissue in less than 2 ms was found to be the same as in PA gels, and thus the same value of ts=2.5 ms as in the previous section was used. Then, volumetric treatments using different spatial step Δz, with values in the range found earlier to be successful in PA gels, and including the case Δz=0, i.e., standard BH, were made.

All volumetric treatments were made according to a methodology previously published by electronically steering the focus over a rectangular grid of 13×5 points, for a total of 65 points, with a spacing of 1 mm between each point in a plane orthogonal to the axis of the BH array. The order in which each point was treated was chosen randomly, as shown in FIG. 5, to minimize the heat accumulation and the risks of prefocal cavitation from a previously treated neighboring points. The acoustic power of the pulses was scaled for each steered focus to reach similar level of the shock amplitude, and thus time-to-boil. Each point in the plane received a steered BH pulse, and the process was repeated until all points received a certain number of pulses (pulses per point, PPP). The pulse repetition frequency (PRF) of the treatment was 1 Hz (resulting in a duty cycle of 1%), and thus the full plane was treated in 65 s. Therefore, from the perspective of one point in the treatment plane, the BH exposure would be like that of previous experiments in PA gel with a steered BH pulse exposure once every 65 s, i.e., the duty cycle for each sonicated point was 0.015%.

After the steered BH treatment, high-resolution U.S. imaging of the samples was done (Sonix RP, Ultrasonix, Richmond, Canada, and L14-5 W probe operating at 14 MHz) in two orthogonal planes. The lesions were recognizable on B-mode images as homogeneous, hypoechoic, rectangular regions, and their sizes (width, height, and depth) were measured. The samples were then cut along the imaging plane (represented as a dash dotted black line in FIG. 7B), and the liquefied tissue inside the lesion was gently washed away using a saline solution. Note that the lesion size measurements were taken from the high-resolution U.S. imaging rather than directly performed on bisected samples, as soft tissue is prone to slight shape changes upon bisection and handling. For each value of Δz, the treatment was considered successful if no solid tissue structures were left in the lesion. If the lesioning was unsuccessful, 2 more steered BH PPP were applied to subsequent lesions with the same value of the Δz parameter.

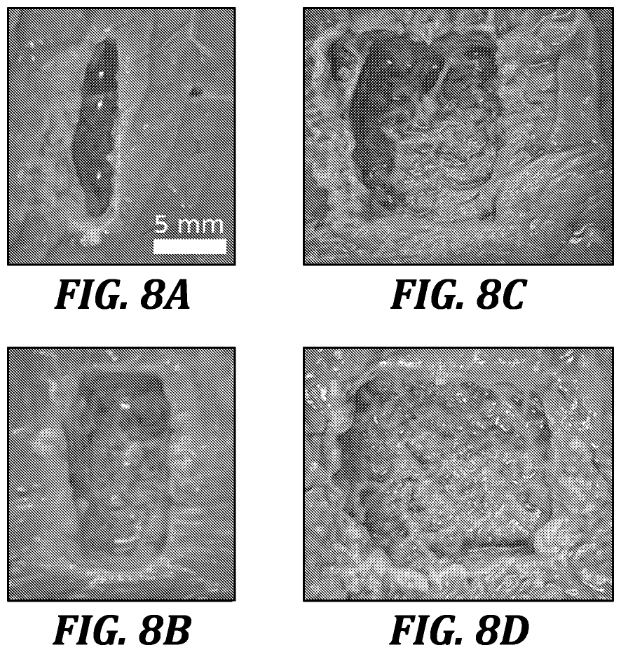

FIGS. 8A-8D are photographs of legion in ex vivo bovine heart tissue using different values of Δz, in accordance with the present technology. Once a value of PPP has been found to yield successful lesions, the treatment was repeated eight times at different locations for the lesion size statistics (n=8). The width and height correspond to the dimensions of the treatment plane (see FIGS. 7A-7B), while the length corresponds to the dimension along the BH transducer axis. The borders of the lesions made with Δz set as 0 and 400 μm were sharp and well defined, and presented thin lightly blanched rim, characteristic of changes in heme iron caused by low temperature increase as observed in previous BH lesions. In contrast, for Δz set as 600 and 800 μm, thin remnants of connective tissue were observed at the borders.

Adding two more PPP to the treatment did not significantly improve the smoothness of the lesion border. More pronounced blanching of the rim was also observable compared to the treatment with lower value of Δz, including some patches of thermal damage as well. All lesions had a notably more irregular distal border, corresponding to the boiling lesion “tails,” as previously reported. The lesion size in the treatment plane was almost the same among all lesions, but its length was significantly affected by varying the parameter Δz. Compared to fixed focus BH, the lesion length in steered focus BH with Δz=400 μm increased by 4.9 mm on average, while further addition of 200 μm of Δz resulted in the increase of the lesion length of almost 3 mm in average, corresponding to the BH focus steering difference. Since larger lesions necessitated more PPP, volumetric ablation rate was calculated for all treatments as overall treatment volume divided by the treatment time. In that regard, steered BH with Δz=400 μm yielded an ablation rate of 4.0 cc/h, for Δz=800 μm it reached 5.5 cc/h-twofold and almost threefold increase compared to fixed-focus BH (2.0 cc/h), respectively.

TABLE 1
Ex Vivo Lesion Dimensions

| Δz | width | height | length | | rate |
|---|---|---|---|---|---|
| (μm) | (mm) | (mm) | (mm) | PPP | (cc/h) |
| 0 | 13.5 ± 0.4 | 6.3 ± 0.3 | 3.4 ± 0.3 | 8 | 2.0 |
| 400 | 13.6 ± 0.7 | 6.5 ± 0.5 | 8.3 ± 0.4 | 10 | 4.0 |
| 600 | 13.3 ± 0.6 | 6.4 ± 0.3 | 11.2 ± 0.5 | 12 | 4.3 |
| 800 | 13.2 ± 0.8 | 6.4 ± 0.3 | 14.0 ± 0.7 | 12 | 5.5 |

Table 1 shows ex vivo lesion dimensions, such as shown in FIGS. 8A-8D. The width and the height correspond to the lesion dimensions in the treatment plane (as shown in FIG. 7A-7B), while the length corresponds to the legion dimension along the BH transducer axis. The mean and standard deviation were obtained from the sample size n=8, and the values for ablation rate were obtained from average lesion dimensions. The length of all the steered BH lesions is statistically significantly different from the standard BG lesions (p<10⁻¹³ per two-tailed Student's t-test) while the other two dimensions are not (p >0.2).

FIGS. 9A-9C are graphs showing acoustic characteristics of the BH pulse in the focal region, in accordance with the present technology. Focal characteristics of the BH pulse at 608 W acoustic power were obtained by acoustic holography and nonlinear modeling. Shown are distributions of peak positive and peak negative pressures: transversely (FIG. 9A) and axially (FIG. 9B). FIG. 2C shows two cycles of the pressure waveform with shocks at the focus.

FIG. 10 is a photograph of steered BH in PA gel with an 800 μm step and a 15 degree angle. FIG. 11 is another photograph of steered BH in PA gel with an 800 μm step and a 15 degree angle. Realization of the steered BH includes a multi-element phased array HIFU transducer with axial steering capability, either a 2-D array having a large number of small elements, as in the current study, or an annular (1-D) array having multiple elements. Such an array may have a driving circuit with the ability to quickly, on the order of microseconds, change the delay on its elements to avoid too long an interruption in the process of emitting a BH pulse. The main goal of this study was to show that a prefocal bubble cloud generated in the standard BH after initiation of boiling can be extended by moving the focus axially toward the transducer. Such discrete movement and generation, step by step, a sequence of proximal additions to the original bubble cloud can lead to higher volumetric ablation rate. This approach therefore enhances some of the mechanisms involved in BH mechanical tissue ablation.

High-speed optical imaging in transparent gels showed that another prefocal bubble cloud can be generated off a previous bubble cloud by shock scattering, effectively extending it. While the first prefocal bubble cloud was generated after boiling started by scattering from the vapor cavity, its further extension was mainly caused by scattering from cavitation cloud formed at each previous step of focus steering. For the following BH pulses, in some cases the full trail of bubbles was seen even without initiation of boiling, as long as bubble nuclei were present at the focus—in our case, remnants of the previous BH pulse. Steering limitations combined with the fixed length of the pulse and fixed temporal step Δt resulted in a maximum spatial step Δz of 800 μm, yielding a 12 mm total steering and a maximum lesion length in PA gels of 15.4 mm, 2.6 times longer than standard BH lesions obtained with the same array without focus steering.

Optical observations also showed a role of the acoustic radiation force in lesion formation in PA gels. For all cases, the gel and bubble clouds were pushed and compressed in the axial direction of the BH transducer, resulting in lesion length longer that the bubble cloud itself. Another observed effect was that the longer in time the area was exposed to the BH pulse, the wider was the final lesion. This resulted from shock-scattering happening not only in the axial direction. In addition, unexpectedly, the bubble cloud itself was pushed in seemingly random directions perpendicular to the BH transducer axis, allowing for the creation of more bubbles on the sides of the cloud.

Axial steering of the HIFU focus during the BH pulse delivery successfully increased the length of volumetric lesions in ex vivo bovine heart, while still allowing single lesions to merge transversely. For all cases of Δz, the length of the lesion in tissue was less than observed in PA gel. This can be explained by the difference between the two media: PA gel is stiff and does not liquefy when exposed to BH pulses, while tissue progressively liquefies—as observed here with incomplete lesions. Once liquefaction starts, other physical mechanisms such as acoustic streaming, microjetting, and atomization inside the vapor cavity, which was not observed in PA gels, take place along with cavitation.

However, as the formation of prefocal bubble clouds plays a role, at least at the start of the treatment, before partial liquefaction of the tissue, PA gel experiments are still important to find and optimize the parameters of the steered BH exposures.

While the volume of lesions in bovine cardiac ex vivo tissue increased with the value of the spatial step Δz, so did the number of pulses required to reach a full liquefaction of the volume targeted. Extra pulses can be used for complete liquefaction of stiff structures inside the tissue, as well as for proper merging each steered BH treatment spot, as it was shown in the PA gel experiments that the width of the bubble cloud was inversely proportional to the spatial step value. Even so, the ablation rate still increased in relation with the value of Δz, up to a maximum of close to threefold for Δz=800 μm. However, the increase of the value of the spatial step and the number of PPP also come with increased visible spread of thermal damages on all borders of the lesion, as well as less-defined borders, especially on the distal side of the lesions where the boiling happened. As cavitational and BH methods typically aim for precise and purely mechanical tissue ablation, these undesired thermal effects, if present, may limit the applications of steered BH. Some BH applications such as hematoma and abscess treatment might benefit greatly from the increased ablation rate of steered BH while its extra thermal effects would not be a concern, whereas the less-defined borders and increased thermal damages might be critical for applications such as precise tissue liquefaction or tumor removal. This would only apply for large values of the spatial step as here the value Δz=400 μm presented no significant thermal effects and well-defined borders of the lesions, and could thus potentially be applied to all standard BH applications.

The method may further be modified by other parameters such as the steering starting time ts, the temporal step Δt, the acoustic power, the shock front amplitude at the focus, and the overall total pulse length. Each of those parameters might impact the ablation rate differently in specific tissues, as well as the thermal effects observed here for large spatial steps.

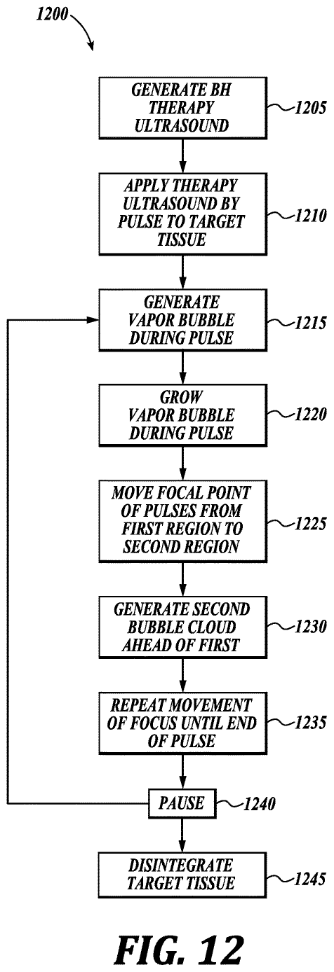

FIG. 12 is an example method 1200 for an ultrasound treatment using high intensity focused ultrasound (HIFU), in accordance with the present technology.

In block 1205, BH therapy ultrasound is generated with a therapy transducer. In some embodiments, the therapy transducer is one that is capable of axially moving a focus of the therapy transducer. In some embodiments, the focus may be moved mechanically or automatically. In some embodiments, the therapy transducer is controlled with a machine, such as a computing device or processor. In some embodiments, the therapy transducer is an array therapy transducer.

In block 1210, the therapy ultrasound is applied by directing a BH ultrasound having ultrasound shock fronts to a target tissue. The target tissue may be any type of organic tissue, including heart tissue, rectal tissue, stomach tissue, muscle tissue, and the like.

In block 1215, at least one μm-scale vapor bubble is generated at the target tissue (as shown and described in detail in FIG. 5A).

In block 1220, the at least one vapor bubble is grown to at least one mm-scale bubble.

In block 1225, a focal point of the pulse is moved from a first region of the target tissue to a second region of the target tissue during the length of the pulse.

In block 1230, a second bubble cloud is generated ahead of the first (as shown and described in detail in FIGS. 5A-5D).

In block 1235, the movements described in block 1225 is repeated until the end of the pulse.

In block 1240, there is a pause between the first pulse and a second pulse. In some embodiments, there may be a plurality of pulses. In such embodiments, the method returns to block 1215 and repeats blocks 1215, 1220, 1225, 1230, 1235, and 1240 for each pulse in the plurality of pulses.

In block 1245, the target tissue is mechanically disintegrated by interactions between the at least one mm-scale bubble and the ultrasound shock waves. In some embodiments, this results in cavitation, such as shown in FIGS. 8A-8D. In some embodiments, the interactions take place within a duration of individual ultrasound pulses of the plurality of ultrasound pulses.

It should be understood that methods 1200 should be interpreted as merely representative. In some embodiments, process blocks of method 1200 may be performed simultaneously, sequentially, in a different order, or even omitted, without departing from the scope of this disclosure.

The present application may reference quantities and numbers. Unless specifically stated, such quantities and numbers are not to be considered restrictive, but representative of the possible quantities or numbers associated with the present application. Also, in this regard, the present application may use the term “plurality” to reference a quantity or number. In this regard, the term “plurality” is meant to be any number that is more than one, for example, two, three, four, five, etc. The terms “about,” “approximately,” “near,” etc., mean plus or minus 5% of the stated value. For the purposes of the present disclosure, the phrase “at least one of A, B, and C,” for example, means (A), (B), (C), (A and B), (A and C), (B and C), or (A, B, and C), including all further possible permutations when greater than three elements are listed.

Embodiments disclosed herein may utilize circuitry in order to implement technologies and methodologies described herein, operatively connect two or more components, generate information, determine operation conditions, control an appliance, device, or method, and/or the like. Circuitry of any type can be used. In an embodiment, circuitry includes, among other things, one or more computing devices such as a processor (e.g., a microprocessor), a central processing unit (CPU), a digital signal processor (DSP), an application-specific integrated circuit (ASIC), a field-programmable gate array (FPGA), or the like, or any combinations thereof, and can include discrete digital or analog circuit elements or electronics, or combinations thereof.

An embodiment includes one or more data stores that, for example, store instructions or data. Non-limiting examples of one or more data stores include volatile memory (e.g., Random Access memory (RAM), Dynamic Random Access memory (DRAM), or the like), non-volatile memory (e.g., Read-Only memory (ROM), Electrically Erasable Programmable Read-Only memory (EEPROM), Compact Disc Read-Only memory (CD-ROM), or the like), persistent memory, or the like. Further non-limiting examples of one or more data stores include Erasable Programmable Read-Only memory (EPROM), flash memory, or the like. The one or more data stores can be connected to, for example, one or more computing devices by one or more instructions, data, or power buses.

In an embodiment, circuitry includes a computer-readable media drive or memory slot configured to accept signal-bearing medium (e.g., computer-readable memory media, computer-readable recording media, or the like). In an embodiment, a program for causing a system to execute any of the disclosed methods can be stored on, for example, a computer-readable recording medium (CRMM), a signal-bearing medium, or the like. Non-limiting examples of signal-bearing media include a recordable type medium such as any form of flash memory, magnetic tape, floppy disk, a hard disk drive, a Compact Disc (CD), a Digital Video Disk (DVD), Blu-Ray Disc, a digital tape, a computer memory, or the like, as well as transmission type medium such as a digital and/or an analog communication medium (e.g., a fiber optic cable, a waveguide, a wired communications link, a wireless communication link (e.g., transmitter, receiver, transceiver, transmission logic, reception logic, etc.). Further non-limiting examples of signal-bearing media include, but are not limited to, DVD-ROM, DVD-RAM, DVD+RW, DVD-RW, DVD-R, DVD+R, CD-ROM, Super Audio CD, CD-R, CD+R, CD+RW, CD-RW, Video Compact Discs, Super Video Discs, flash memory, magnetic tape, magneto-optic disk, MINIDISC, non-volatile memory card, EEPROM, optical disk, optical storage, RAM, ROM, system memory, web server, or the like.

The detailed description set forth above in connection with the appended drawings, where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to be exhaustive or to limit the disclosure to the precise forms disclosed. Similarly, any steps described herein may be interchangeable with other steps, or combinations of steps, in order to achieve the same or substantially similar result. Generally, the embodiments disclosed herein are non-limiting, and the inventors contemplate that other embodiments within the scope of this disclosure may include structures and functionalities from more than one specific embodiment shown in the figures and described in the specification.

In the foregoing description, specific details are set forth to provide a thorough understanding of exemplary embodiments of the present disclosure. It will be apparent to one skilled in the art, however, that the embodiments disclosed herein may be practiced without embodying all the specific details. In some instances, well-known process steps have not been described in detail in order not to unnecessarily obscure various aspects of the present disclosure. Further, it will be appreciated that embodiments of the present disclosure may employ any combination of features described herein.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

While illustrative embodiments have been illustrated and described, it will be appreciated that various changes can be made therein without departing from the spirit and scope of the technology.

1. A method for an ultrasound treatment using high intensity focused ultrasound (HIFU), the method comprising:
generating boiling histotripsy (BH) therapy ultrasound with a therapy transducer;
applying the therapy ultrasound by directing a plurality of ultrasound pulses having ultrasound shock waves to a target tissue;
generating at least one μm-scale vapor bubble at the target tissue within each pulse;
growing the at least one vapor bubble to at least one mm-scale bubble;
moving a focal point during each pulse of the plurality of pulses from a first region of the target tissue to a second region of the target tissue; and
mechanically disintegrating the target tissue by interactions between the ultrasound shock waves with the at least one mm-scale bubble and bubble clouds, wherein the interactions take place within a duration of individual ultrasound pulses of the plurality of ultrasound pulses.

2. The method of claim 1, wherein the plurality of pulses has a duration of 1-20 ms.

3. The method of claim 1, wherein the therapy transducer operates in a frequency range of 1 MHz to 20 MHz.

4. The method of claim 1, wherein the therapy transducer is moved between a plurality of regions, including the first region and the second region.

5. The method of claim 1, wherein the second region is closer to the therapy transducer than the first region.

6. The method of claim 1, wherein a shock amplitude at the focal point of the therapy transducer is in a range of 40 MPa to 250 MPa.

7. The method of claim 1, wherein the plurality of ultrasound pulses is applied at a focal depth of 0.25 cm to 15 cm.

8. The method of claim 1, wherein the focal point is moved axially from the first region to the second region.

9. The method of claim 1, wherein the focal point is moved at an angle.

10. The method of claim 9, wherein the angle is about 0 to 35 degrees.

11. The method of claim 1, wherein a Δz of the movement of the focal point ranges from 100 to 800 μm.

12-22. (canceled)

23. A method for an ultrasound treatment using high intensity focused ultrasound (HIFU), the method comprising:
generating boiling histotripsy (BH) therapy ultrasound with a therapy transducer;
applying the therapy ultrasound by directing a plurality of ultrasound pulses having ultrasound shock fronts to a target tissue;
generating at least one μm-scale vapor bubble at the target tissue during each pulse;
growing the at least one vapor bubble to at least one mm-scale bubble;
moving a focal point of each pulse of the plurality of pulses between a plurality of regions of the target tissue; and
mechanically disintegrating the target tissue by interactions between the ultrasound shock waves with the at least one mm-scale bubble and bubble clouds, wherein the interactions take place within a duration of individual ultrasound pulses of the plurality of ultrasound pulses.

24. The method of claim 23, wherein the plurality of regions is an array of regions corresponding to the target tissue.

25. The method of claim 24, wherein the method further comprises:
sequencing each region in the array of regions; and
moving the focal point between each region of the array based on the sequence.

26. The method of claim 25, wherein the method further comprises:
applying a number of pulses at each region of the array of regions, wherein the number of pulses differs between each region of the array of regions.

27. The method of claim 23, wherein the focal point is moved axially.

28. The method of claim 23, wherein a shock amplitude at the focal point of the therapy transducer is in a range of 40 MPa to 250 MPa.

29. The method of claim 23, wherein the plurality of ultrasound pulses is applied at a focal depth of 0.25 cm to 15 cm.

30. The method of claim 23, wherein the focal point is moved at an angle.
